(19) United States
(12) Patent Application Publication
(10) Pub. No.: US 2025/0325796 A1
(43) Pub. Date: Oct. 23, 2025

(54) LIQUID DELIVERY DEVICE WITH A ONE-WAY VALVE

(71) Applicant: Merxin Limited, King's Lynn (GB)

(72) Inventors: Graham PURKINS, King's Lynn (GB); Stephen HOWGILL, Leicester (GB); Adam STUART, King’s Lynn (GB)

(73) Assignee: Merxin Limited, King's Lynn (GB)

(21) Appl. No.: 19/199,858

(22) Filed: May 6, 2025

Related U.S. Application Data
(63) Continuation-in-part of application No. PCT/IB2024/060702, filed on Oct. 30, 2024.

(30) Foreign Application Priority Data
Oct. 30, 2023 (GB) 2316588.9

(51) Int. Cl. A61M 39/24; A61M 11/00

(52) U.S. Cl. CPC A61M 39/24; A61M 11/003; A61M 2039/2413; A61M 2039/2466

(57) ABSTRACT

A liquid delivery device has a lower housing containing a reservoir and an upper housing containing a nozzle assembly with an upper tube housing, a passage formed through the upper tube housing between the reservoir and the nozzle assembly, the passage within the upper tube housing forming a metering chamber; the nozzle assembly converting liquid from the metering chamber to a droplet spray; the liquid delivery device having a one-way valve that locates and seals within the passage; the valve configured so that in a first state fluid flow through the passage is substantially blocked, and in a second state fluid can flow through the passage; the valve deforming from the first state to the second state, the upper tube housing comprising a recess at the upper end, the recess configured to receive and confine the outer side of the one-way valve.

### TECHNICAL FIELD

The aspects of the disclosed embodiments relate to a liquid delivery device with a one-way valve. More particularly, the aspects of the disclosed embodiments relate to a nebuliser comprising a nozzle assembly with a one-way valve. Even more particularly, the aspects of the disclosed embodiments relate to a drug delivery device comprising a nozzle assembly with a one-way valve.

### BACKGROUND

Drug delivery devices such as nebulisers are used to produce an aerosol of droplets for inhalation through the mouth and pharyngeal cavity into the lungs of a patient, for nasal administration, or for spraying the surface of the eye.

In a nebulising drug delivery device such as a soft mist inhaler (SMI), liquid pharmaceutical formulations are typically stored in a reservoir. It is common in known types of SMI devices to use a collapsible reservoir, formed with a stiff outer shell, and containing a flexible ‘bag’ formed from a flexible plastic material. Other types of reservoir are also used, such as rigid or semi-rigid containers, or other alternative reservoir collapsible or non-collapsible structure.

In use, the liquid pharmaceutical formulations are conveyed from the reservoir through a riser tube into a pressure chamber, and then forced through a nozzle under pressure and atomised. In this way, drug delivery devices such as SMIs are able to nebulise a small amount of a liquid formulation according to the required dosage within a few seconds, to produce an aerosol suitable for therapeutic inhalation. Moreover, this can be achieved without requiring the use of a propellant, delivering an environmental benefit given the undesirable impact of propellants in climate change.

A typical, known, type of SMI or nebuliser device 1 is shown in FIG. 1. The device 1 has an upper half 2 that contains a nozzle assembly (the ‘nozzle’ of the nozzle assembly is generally formed by the upper conical surface of the nozzle retainer 15), and a lower half 3 that contains a reservoir that includes a ‘collapsible bag’ of the type referred to above. The device 1 is configured so that a passage is formed between the reservoir and the nozzle. The upper and lower halves 2, 3, are in use rotated relative to one another to pump or prime the device 1. As the device 1 is pumped (by rotating the two halves 2, 3, relative to one another), the riser/capillary tube 6 is pulled downwards (that is, away from the filter holder 10 as shown in the variation of device 1 shown in FIG. 6, and as shown in the difference between the two positions of the capillary tube 6 in the variation of device 1 shown in FIGS. 9a and 9b. This movement of the capillary tube 6 increases the volume of the metering chamber 24, so that the pressure in the metering chamber 24 drops to lower than atmospheric pressure. This lower pressure is ‘applied’ to the reservoir as suction, so that liquid is pulled from the reservoir, into the capillary tube 6, and towards the nozzle, and the ‘collapsible bag’ structure that forms part of the reservoir is pulled inwards (collapses inwards). For each individual use, the collapse is small, with each use adding incrementally to the overall full collapse of the bag at end-of-life.

Once the device has been primed, the device can then be triggered by a user in order to deliver a dose of product. When the device is triggered by a user, a nebulised mist of product is delivered through the nozzle assembly at the upper end of the device.

In devices of this type, there is an inherent resistance to reservoir collapse/reduction. For example, in a reservoir of the type that has a collapsible bag within a stiffer outer shell, even with a collapsible bag that has a thin wall, there is an inherent resistance to collapse/reduction, and this resistance increases as the bag empties. The bag is always ‘fighting’ the collapse caused by suction of the contents from the bag. The more resistance to collapse, the more resistance there is to drawing liquid up. Therefore, a large pressure differential is important in order to effectively cause the suction effect.

However, nozzle assemblies of this type can create an air leakage path from the exterior of the device to the interior. When the device is pumped or primed, liquid from the bag is drawn into and up the tube as outlined above, but air can also be drawn inwards through the nozzle assembly, or by bypassing seals around the nozzle assembly. This impacts on the pressure differential created by pumping/priming the device, which impacts on the effectiveness of liquid being drawn from the bag to fill the chamber, and results in a reduction of the ability of the device to deliver a consistent or repeatable dose. The dead space in the micropump also impacts the maximum achievable pressure differential during priming, with a larger dead space reducing the pressure differential that can be created and thus reducing the ability to draw liquid from the bag and collapse the bag.

The air leakage path also provides an open route through the nozzle assembly. Due to the open access to the environment, this can reduce the shelf-life, stability, and protection of the formulation.

The use of one-way valves to assist with preventing air leakage into an inhaler or similar device is known.

U.S. Pat. No. 9,050,428 describes and shows a device, cartridge and method for dispensing a liquid. The liquid is pressurised in a cartridge to a first lower pressure and, then, pressurised in doses by a pump to a second higher pressure. A valve is located between the cartridge and the pump, and is normally closed and/or opened only temporarily. Thus, evaporation and dripping of liquid can be avoided or minimized.

US2013/200110 describes and shows a metering device for metered dispensing of a fluid, a storage container shown connected to a metering head, a spindle being guided in the metering head and having a through-channel for the fluid to be transported.

In this specification where reference has been made to patent specifications, other external documents, or other sources of information, this is generally for the purpose of providing a context for discussing the features of the disclosed embodiments. Unless specifically stated otherwise, reference to such external documents is not to be construed as an admission that such documents, or such sources of information, in any jurisdiction, are prior art, or form part of the common general knowledge in the art.

### SUMMARY

The aspects of the disclosed embodiments are directed to provide a liquid delivery device comprising a nozzle assembly with a one-way valve which goes some way to overcoming the abovementioned disadvantages or which at least provides the public or industry with a useful choice.

Further aspect of the disclosed embodiments are directed to providing a nebuliser comprising a nozzle assembly with a one-way valve which goes some way to overcoming the abovementioned disadvantages or which at least provides the public or industry with a useful choice.

Still further aspect of the disclosed embodiments are directed to providing a drug delivery device comprising a nozzle assembly with a one-way valve which goes some way to overcoming the abovementioned disadvantages or which at least provides the public or industry with a useful choice.

Accordingly, in a first aspect the aspects of the disclosed embodiments may broadly be said to consist in a one-way valve for a liquid delivery device, comprising: a main body configured to locate and seal within a passage within the liquid delivery device; a sealing portion configured so that in a first state fluid flow through the passage is substantially blocked, and in a second state fluid can flow through the passage; the main body and sealing portion comprise parts of a unitary item, the sealing portion configured to deform from the first state to the second state in use, the main body configured so as to remain substantially in position and substantially undeformed,

In an embodiment, the valve further comprises a plurality of connecting members configured to extend between the main body and the sealing portion to bias the blocking member towards the first position, a plurality of apertures formed between the connecting members, the apertures blocked in the first state and open in the second state.

In an embodiment, the main body comprises a ring-shaped body and the central sealing portion comprises a disc-shaped member, the main body substantially surrounding and enclosing the sealing portion, the central sealing portion sized to substantially fully fill the lower end of the ring-shaped main body.

In an embodiment, the central sealing portion has a height or thickness substantially one-third the height of the main body.

In an embodiment, the lower face/side of the central sealing portion is substantially aligned with and in the same plane as the lower end of the main body.

In an embodiment, the upper and lower faces of the disc-shaped member are substantially flat and parallel to one another.

In an embodiment, the connecting members comprise four Y-shaped pillars that extend in between and connect between the central sealing portion and the ring-shaped main body, the pillars located at equally-spaced intervals around the inside of the main body.

In a second aspect the disclosed embodiments may broadly be said to consist in a liquid delivery device, comprising: a lower housing part configured to contain a reservoir; an upper housing part configured to contain a nozzle assembly, the nozzle assembly comprising an upper tube housing, a passage extending between the reservoir and the nozzle assembly to in use deliver liquid from the reservoir to the nozzle assembly, at least part of the passage formed through the upper tube housing, the upper part of the passage within the upper tube housing forming a metering chamber; the nozzle assembly configured to convert liquid received from the metering chamber to a droplet spray at the outer or downstream end of the nozzle assembly for delivery to a user; the liquid delivery device further comprising a one-way valve according to any one of the preceding statements; the upper tube housing comprises a recess at the upper end of the upper tube housing, the recess configured to receive and substantially fully confine the outer side or sides of the one-way valve.

In an embodiment, the valve is located substantially directly at the upper end of the metering chamber.

In an embodiment, the recess of the upper tube housing and nozzle assembly are configured so that the end of the nozzle assembly locates into the recess.

In an embodiment, the valve and nozzle assembly are further mutually configured so that the valve is confined within the recess by contact with the nozzle assembly.

In an embodiment, the liquid delivery device further comprises a spring configured to provide pressure to liquid within the metering chamber, the valve configured to deform to allow substantially 80 Cubic Micrometres to pass therethrough.

In an embodiment, the one-way valve and nozzle assembly are mutually configured so that the one-way valve only opens by an area substantially the same as the nozzle jet area/nozzle cross sectional area.

Therefore, the foregoing is considered as illustrative only of the principles of the invention.

Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### BRIEF DESCRIPTION OF THE DRAWINGS

One or more embodiments of the present disclosure will now be described, by way of example only, with reference to the accompanying drawings, in which:

FIG. 1 shows a side view of a known, prior art type of manually-operated nebuliser that has an upper half and a lower half, the two halves rotated relative to one another in use to pump or prime the device for use.

FIG. 2 shows a side perspective view of the nebuliser of FIG. 1, the lid of the nebuliser shown partly open.

FIG. 3 shows a perspective top view of the nebuliser of FIGS. 1 and 2, showing detail of a mouthpiece that is enclosed within the lid when the lid is closed, and the top part of a nozzle assembly that is enclosed within the mouthpiece.

FIG. 4 shows a cross-sectional side perspective view of the nebuliser of FIGS. 1 to 3, showing detail of the nozzle assembly, mouthpiece and other associated elements, and detail of a capillary tube that leads between the nozzle assembly and a liquid reservoir.

FIG. 5a shows a perspective top view of the nozzle assembly of the nebuliser of FIGS. 1 to 4.

FIG. 5b shows a cutaway side view of the nozzle assembly of FIG. 5a from the same angle as FIG. 5a, showing internal detail of the nozzle assembly.

FIG. 6 shows a stylised cutaway side view of the nozzle assembly of the nebuliser of the preceding figures.

FIG. 7a shows a cutaway side view of a known prior art type of valve,, showing an example of the amount of ‘dead space’ created by using a valve of this type.

FIG. 7b shows a cutaway side view of a known prior art type of valve,, showing an example of the amount of ‘dead space’ created by using a valve of this type

FIG. 8 shows a cutaway side view of a nozzle assembly according to an embodiment of the disclosed disclosure, showing detail of a one-way valve that forms part of the nozzle assembly.

FIGS. 9a and 9b show cutaway side views of the nozzle assembly of FIG. 8, showing detail of the change in position of a capillary tube that forms part of the nozzle assembly as the device is pumped or primed ready for use.

FIGS. 10a and 10b show top perspective and top views respectively of the one-way valve of FIGS. 8 and 9, according to an embodiment of the disclosure.

FIG. 11 shows a side perspective cutaway view of the one-way valve of FIGS. 10a and 10b.

FIG. 12 shows a perspective top view of an upper tube housing according to an embodiment of the disclosure, and as shown as part of the nozzle assembly of FIGS. 8 and 9, the upper tube housing configured so as to have a recess formed in the top end of the upper tube housing, the recess retaining the one-way valve.

FIG. 13 shows a cutaway side view of the top end of a nebuliser according to an embodiment of the present disclosure, showing detail of the fluid flow path around the seal in use.

### DETAILED DESCRIPTION

Detailed embodiments of the disclosure will now be described with reference to the figures.

### General

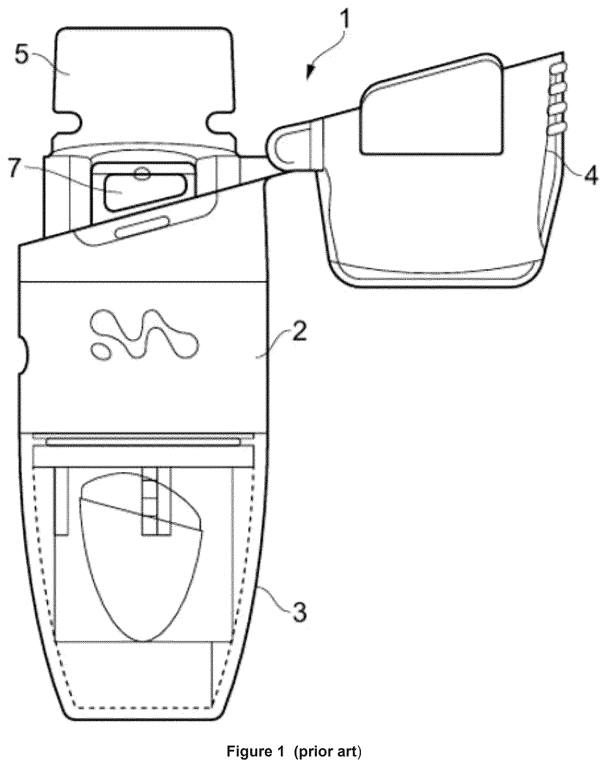

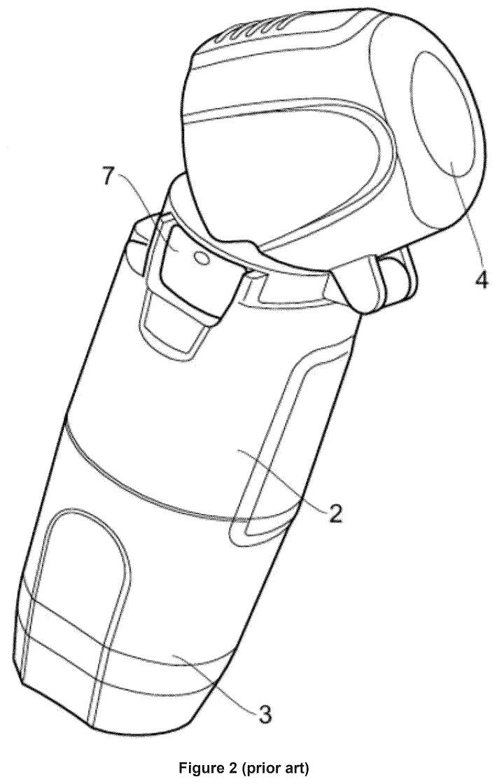

The one-way valve 150 of the present disclosure is described below as being used as part of a liquid delivery device—specifically, a nebuliser device. A prior art nebuliser 1 is shown in FIGS. 1 and 2, and the liquid delivery device of the present disclosure is similar to this. There is some commonality of components between the prior art nebuliser 1 and the nebuliser of the present disclosure. The internal changes and differences of the components are as detailed below.

References to orientations such as ‘upwards’, ‘downwards’ and similar should be taken in this specification as meaning the orientation shown in FIG. 1 (i.e. the nebuliser stood upright as if on a horizontal surface such as a table top), even if in use the orientation would differ from this. Similar numbering will be used to refer to similar elements on all the devices as described below—e.g. nebuliser device 1 for the general, known/prior art nebuliser, and nebuliser device 100 for the device of the present disclosure. Similarly, filter holder 9 for the general form, and filter holder 109 for the filter holder as used in nebuliser 100, etc.

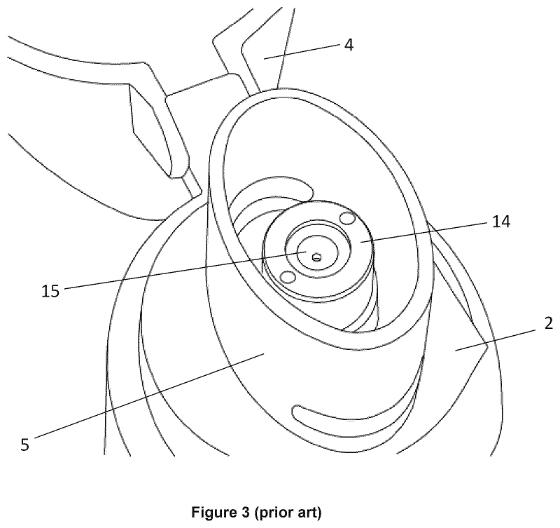

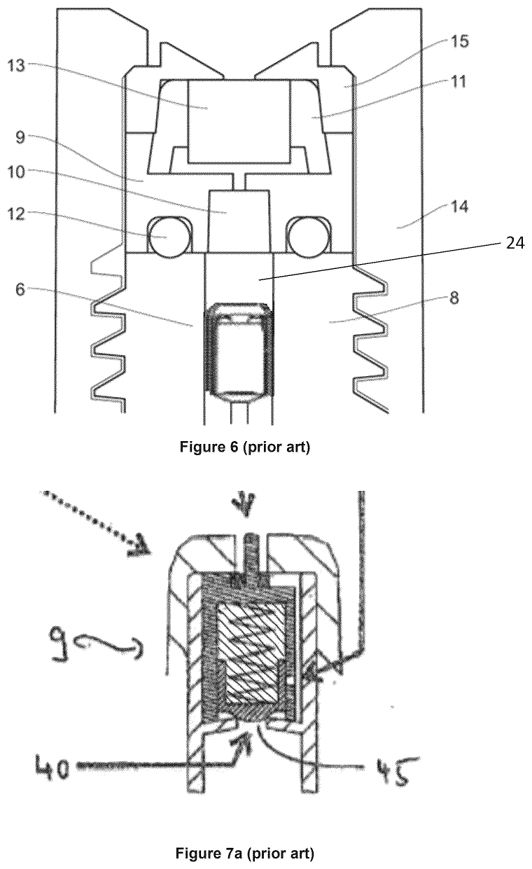

The prior art nebuliser contains a nozzle assembly, as shown in FIG. 4. A general or stylised form of nozzle assembly, and other relevant parts, are shown in FIG. 6 for the purposes of illustration of the general structure.

The general form of nebuliser such as is known in the art comprises an upper housing (generally designated as ‘2’) that contains a nozzle assembly (the parts that form the nozzle assembly shown in FIG. 6), and a lower housing (generally designated as ‘3’) that in use contains a collapsible reservoir or ‘bag’ (not shown in the figures). A capillary tube 6 extends between the bag and the nozzle assembly.

The nozzle assembly comprises: a filter holder 9; a filter 10; a nozzle seal 11; a nozzle chip/filter 13, and; a nozzle holder or nozzle retainer 15. The elements that form the nozzle assembly are retained on an upper tube housing 8 with a top nut or top cap 14, with a lower seal 12 sealing between the upper tube housing 8 and the filter holder 9. The filter holder 9, filter 10, nozzle seal 11, lower seal 12, and nozzle chip/filter 13, nozzle holder 15, are all enclosed within the top nut 14 once assembled. In use, the top nut 14 screws onto the upper end of the upper tube housing 8 that is located within the upper housing part 2. The upper tube housing 8 has a passage passing substantially axially therethrough, with a capillary tube 6 passing most of the way through the passage from the base or lower end of the nebuliser 1 (that end that is towards the bag), to the upper end where the nozzle assembly is located. The space between the upper end of the capillary tube 6 and the lower end of the filter 10 forms a metering chamber 24.

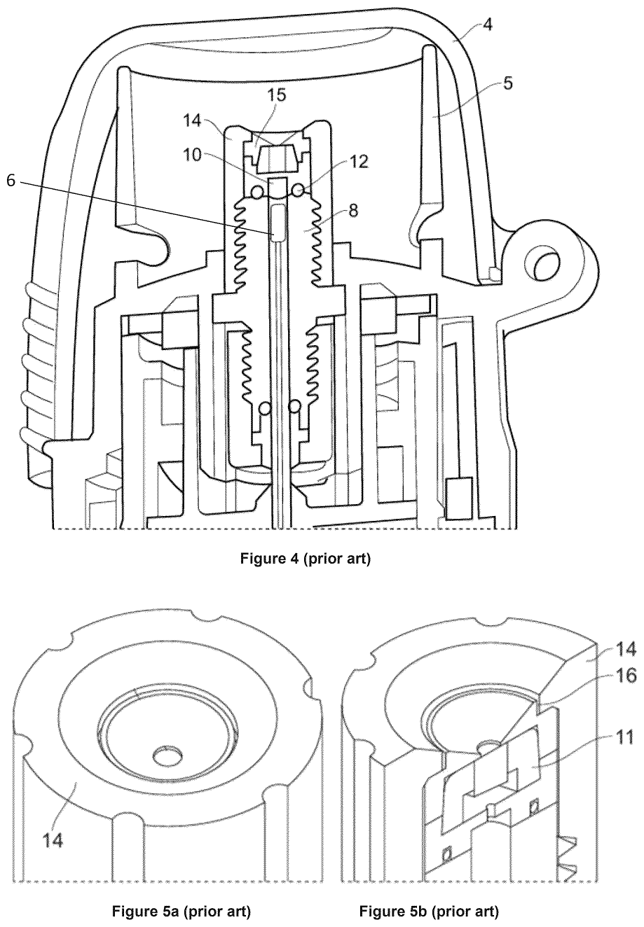

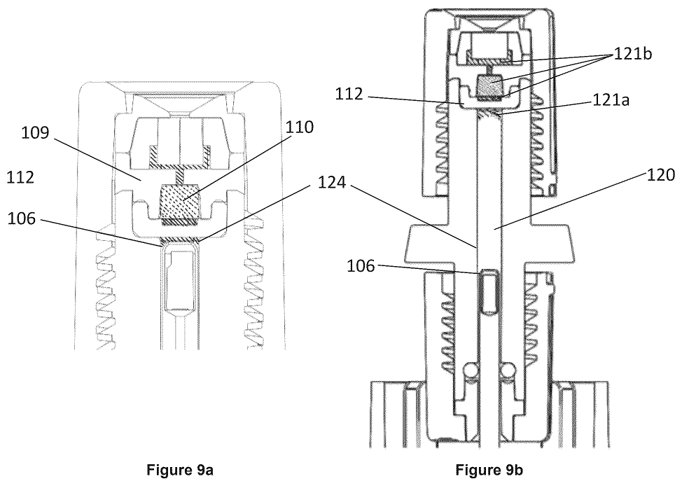

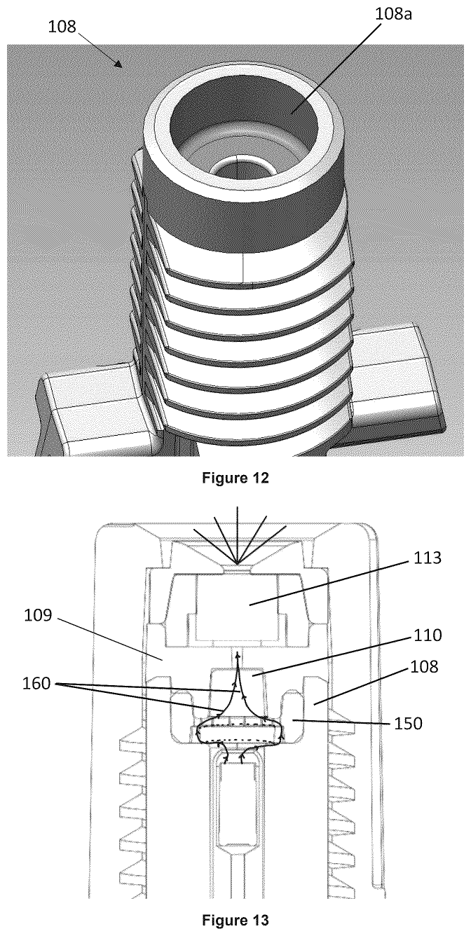

The nebuliser 100 of the present disclosure has substantially similar elements to those described above and as shown in FIGS. 4 to 7. However, the nebuliser 100 of the present disclosure comprises an upper tube housing 108 that differs from the known type of upper tube housing 8 that is shown in FIGS. 4-7 and which is shown as upper tube housing 108 in FIG. 12, and the nebuliser 100 further comprises a one-way valve 150. The one-way valve 150 is located in a recess 108a at the top end the upper tube housing 108, so that when the nebuliser 100 is assembled, the one-way valve 150 is located at the top end of the metering chamber (the metering chamber numbered as 124 in FIGS. 8 and 9). That is, the one-way valve 150 is located above (i.e. downstream of), the space that forms the metering chamber 124.

In the embodiment shown and described, the upper tube housing 108 differs from the upper tube housing 8 of the prior art device 1, with the upper tube housing 108 configured so as to have a recess 108a formed in the top end of the upper tube housing 108. The valve 150 is retained in recess 108a, directly underneath the filter (filter 110 in this embodiment). As can be seen, the valve 150 and upper tube housing 108 are configured so that the sides of the valve 150 are fully confined when the valve 150 is located in the recess 108a.

In use, the capillary tube 106 moves within the passage towards and away from the nozzle assembly, as the nebuliser device is used. For the purposes of this specification, the metering chamber 124 should be considered to run between the lower side or end of the one-way valve 150 and the top or inner end of the capillary tube 106. It can be seen that the volume of the metering chamber 124 will change with movement of the capillary tube (similar to the difference as shown in FIGS. 9a and 9b), the volume being greatest when the capillary tube is located at its furthest ‘downwards’ extent—i.e. the position the capillary tube is in when the inhaler is fully cocked and ready to dispense a dose. That is, furthest ‘downwards’ when the inhaler is in the orientation shown in FIG. 1, which is the orientation for a nebuliser standing upright.

In use, a user rotates the upper and lower parts relative to one another to pump or prime the device for use. That is, to ‘cock’ the device. This action causes the capillary tube to move away from the one-way valve 150 (that is, downwards towards the base of the nebuliser if it is aligned upright in a similar manner to the prior art nebuliser shown in FIG. 1) to its lowest position. This lowers the pressure inside metering chamber 124 (the volume of metering chamber 124 is increased without there being a corresponding increase in its contents). This causes liquid from the bag to be sucked up the capillary tube to the upper end, the liquid exiting the upper end of the tube into the metering chamber 124. That is, before the device is primed, the metering chamber 124 and the bag are at substantially the same internal pressure. When the pressure in the metering chamber decreases, liquid is sucked up the tube from the higher pressure location (the bag)—to the lower pressure region (chamber 124) until enough liquid has flowed to the chamber for the pressure to substantially equalise.

This relative rotational action of the upper and lower housing parts also compresses a spring (not shown) within the nebuliser. When a user then triggers the dispensing mechanism (e.g. by pressing a button or similar on the housing of the nebuliser—button 7 on the prior art nebuliser, as shown in FIG. 1), the tube is forced upwards by the release of compression in the spring, and the head of the tube moves rapidly upwards along the passage, forcing liquid out of the metering chamber 124 though the one-way valve 150, and then through the nozzle assembly as a spray of fine droplets.

### One-Way Valve

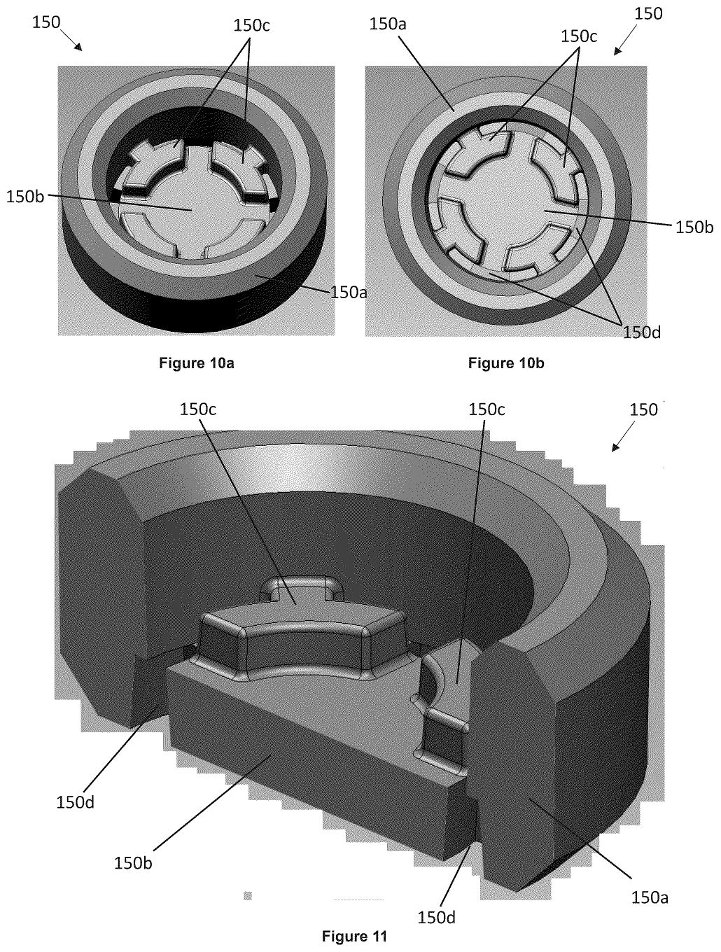

As shown in FIGS. 10 and 11, the one-way valve 150 of this embodiment of the present disclosure comprises a unitary member formed from silicone rubber or similar. The valve 150 comprises a ring-shaped main body 150a (circular in plan view), a central sealing portion 150b, and connecting members 150c that connect between the sealing portion 150b and the main body 150a. The ring-shaped main body 150a runs circumferentially around the outside of the sealing portion 150b and connecting members 150c to enclose these. The sealing portion 150b and connecting members 150c are located at the lower side or end of the main body 150a.

The main body 150a has a lower end with a flat inner portion and an outwardly-chamfered or outer edge. The upper end has a flat central section, with chamfered inwards and outwards edges, the outer chamfer larger than the inner chamfer.

The main body 150a acts to hold and stabilise the one-way valve 150 in position on/in the recess 108a formed in the top of the upper tube housing 108, the recess 108a configured to receive the one-way valve 150.

The central sealing portion 150b comprises a disc-shaped member, circular in plan view, having a thickness approximately one-third the height of the main body 150a. The upper and lower faces of the disc are substantially flat and parallel to one another. The lower face/side of the central sealing portion 150b is substantially aligned with and in the same plane as the flat inner portion of the lower end of the main body 150a.

The connecting members 150c comprise four pillars, Y-shaped in plan view, that extend in between and connect between the central sealing portion 150b and the main body 150a. The pillars are located at equally-spaced intervals around the inside of the ring-shaped main body 150a, towards the lower end. The arms of the Y-shaped pillars are curved so as to form a broken circle (a circle with a broken perimeter) around the top of the central sealing portion 150b, with a small gap between the arms and the surface of the inner wall of the ring-shaped main body 150a. That part of the central sealing portion within/covered by the circle formed by the pillars comprises a perimeter part of the central sealing portion, the part within the perimeter of the circle forming a central part of the central sealing portion.

The central sealing portion 150b is sized so as to fully fill the lower end of the ring-shaped main body 150a, except for four apertures 150d that are formed between the inner surface of the main body 150a and the outer side edge of the disc of the central sealing portion 150b.

The apertures 150d are formed so as to follow the curve of the gap between the arms and the surface of the inner wall of the ring-shaped main body 150a, and are sized (in plan view) so as to fill the space between adjacent ends of the pillars on the connecting members 150c, overlapping slightly with the ends of the pillars, as shown in FIG. 10b.

The one-way valve 150 provides a seal in normal use, but the central section 150b deforms under pressure to allow fluid flow from the reservoir to the nozzle assembly through the apertures 150d. The valve 150 effectively acts as a seal whilst the device is at rest, the one-way valve 150 acts to shut off the channel from the metering chamber to the nozzle, and acts to prevent air from being pulled into the metering chamber 124 via the nozzle assembly during priming of the device.

When upwards pressure is applied to the central sealing portion 150b (high pressure in the metering chamber 124, caused by the tube being forced upwards by the release of compression in the spring), the central section 150b deforms (that is, the shape or profile that the central section 150b has in a condition when no pressure is being applied, distorts or changes when pressure is applied), and will tend to return to an undeformed state when external forces (the upwards pressure) is removed. The centre of the central sealing portion 150b is free to deform upwards, while the ring-shaped main body 150a is constrained by the rigid walls of the upper tube housing 108. The central sealing portion 150b is not constrained in a similar fashion. As the central section 150b deforms from a first (undeformed) state to a second (deformed) state, this opens the apertures 150d.

As can be seen, the connecting members 150c connect to and at least partly extend over the perimeter part of the central sealing portion 150b. The pillars do not extend over the top of the central part of the central sealing portion 150b in a manner that would substantially prevent or meaningfully inhibit movement and/or deformation of the central part, although it is possible that an immaterial portion of the structure of the arms could potentially extend at least partly into the central section.

### Operation

As noted above, when a user rotates the upper and lower parts of the inhaler relative to one another to pump or prime the device for use this causes a pressure drop in the metering chamber 124. The volume of the space that forms the metering chamber 124 increases, while the same amount of fluid/gas is present. As the pressure on the upper side of the one-way valve 150 doesn't change, this causes a pressure difference that pushes on the valve 150 and causes a tighter seal between the one-way valve and the upper tube housing 108. This helps to increase the quality of the seal. A good/effective seal is highly desirable, as air-ingress through the nozzle past the valve 150 and into the metering chamber 124 causes liquid displacement, and can cause variation in the completeness of filling and subsequent delivered volume of liquid on the next firing stroke—that is, when a user dispenses a dose. Variation on the completeness of filling can cause unacceptable variations in the size of the dose delivered. Further, air ingress reduces the pressure difference between the metering chamber 124 and the bag during the priming action when the inhaler upper and lower parts are rotated relative to one another to pump or prime the device for use. This reduces that amount of pressure available to draw liquid from the reservoir, increases the time required to prime/fill the metering chamber 124, and can cause an incomplete or reduced dose to be delivered to the chamber 124 (and then delivered to a user). Therefore, an effective seal that helps to prevent air ingress is highly desirable. A high-pressure difference also assists with the collapse of the cartridge reservoir—the reservoir collapses inwards as liquid is drawn out of the reservoir and into the metering chamber 124, and a high pressure difference assists with this process by helping to overcome the resistance of the reservoir to collapse.

It can be seen that a reduction in the pressure difference caused by air leakage is undesirable.

As described above, and as shown in FIGS. 9a and 9b, the capillary tube 106 (that forms a piston) moves in use between a rest position (as shown in FIG. 9a), and a primed position (as shown in FIG. 9b) when the capillary tube 106 is pulled downwards (swept back). During this action, the metering chamber 124 increases in volume. This volume (the increase in volume created by the capillary tube being drawn back on priming) is the swept volume. The swept volume is constant, in order to deliver a consistent dose. The swept volume is shown in FIG. 9b, and designated as ‘swept volume 120’.

However, in an inhaler such as the known type of inhaler 1 and also the inhaler of the embodiment of the present disclosure, there is a volume that is not part of the swept volume, but is available to be filled with liquid. This may include, for example, the volume created between the inside diameter of the housing and the outer diameter of the capillary tube, and areas of volume capable of holding liquid (i.e. that volume that is fully open or which is not fully solid) between the end of the capillary tube at rest and the nozzle exit. This volume includes (part of) the filter (filter 10 for the known type of inhaler, and filter 110 for the embodiment of the present disclosure), which is porous and therefore has a liquid volume as part of the overall volume of the filter (the overall volume of the filter being both of the liquid portion and the solid portion that forms the structure of the filter—in liquid delivery devices of this type, the liquid volume of the filter is substantially between 30% and 55% of the overall volume of the filter). These volumes create a dead space. The total volume comprises the swept volume, plus the volume of the dead space of the inhaler.

The dead space can be divided into the dead space below the valve (shown in FIGS. 9a and 9b and designated as ‘121a’), and the dead space above the valve (shown in FIGS. 9a and 9b by cross-hatched lines, and the dotted cross-hatched lines over the filter 110, and designated as ‘121b’).

On priming, the ratio of swept volume to total volume influences the effectiveness of the device to prime. Thus, the ratio of swept volume to total volume influences the amount of pressure difference or partial vacuum. It can therefore be seen that the ratio of swept volume to total volume is dependent on the total volume, as the swept volume is constant. The ‘level’ of vacuum drops off rapidly with changes to this ratio—e.g. a total volume of 30 cubic millimetres (15 cubic millimetres of swept volume plus 15 cubic millimetres of space between the nozzle assembly exit and the top of the one-way valve 124) gives a ratio of swept volume:total volume of 1:2, limiting the available pressure difference to half an atmosphere.

Wherever the piston stops at the end of an upwards/operation stroke, to the end of the nozzle, is a volume that is referred to as ‘dead space’. The amount of dead space has a significant influence on how well the device will prime—too much dead space and the device will not prime. Also, the more dead space there is to be filled, the more initial priming shots are required in order to ‘bed in’ the device and for it to reach a stage where in use a consistent shot weight is produced.

A vacuum is required to draw liquid up the capillary tube. The capillary tube is required to be a certain diameter in order for liquid to be drawn up the tube (the diameter and other dimensions have to be within certain limits), and it is therefore highly beneficial that the pressure drop is as large as possible. A large pressure drop also assists with overcoming the resistance of the bag and bottle to collapse (collapse is required in use so that liquid is drawn out of the bag and bottle).

It can be seen that to maximise the pressure drop and to minimise the pre-fill volume (that is, the swept volume 120 plus the below-the-valve dead space/pre-valve dead space 121a—the volume needed to completely fill the metering chamber (pre-valve) and have full suction on subsequent actuations), the volume between the top of the piston 106 in the metering chamber and the exit of the nozzle assembly should ideally be zero. However, it can also be seen that this is not practically possible as there are other functionality considerations, and certain components are required to achieve these. For example a filter such as filter 110 provides robustness advantages, but adds volume and therefore dead space. Where a one-way valve is added to prevent leakage during storage, it may increase dead space. This dead space should be minimised to avoid negatively impacting device performance.

What is required of a one-way valve is functionality but with minimised negative impact on product performance. Creating components that minimise the amount of dead space is advantageous in achieving the beneficial effects of the valve, whilst minimising undesirable secondary negative impacts on product performance.

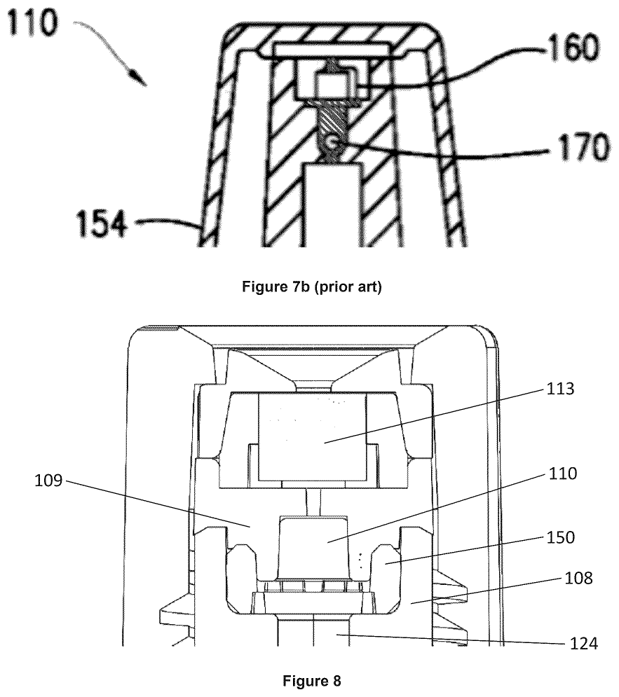

Prior art one-way valves such as those discussed in the ‘background’ section will often use a mechanism that creates a very large volume of dead space. For example if the one-way valve uses e.g. a ball-and-spring seal (a ball held in position in an aperture by a coil spring), the spring creates a large area of dead space around the coil. This is shown for example in FIG. 7a, which is based on FIG. 8 of US2013/200110, and FIG. 7b, which is based on FIG. 3 of U.S. Pat. No. 9,050,428. The cross-hatching added to these figures shows the ‘dead space’ created by using a ball-and-spring valve.

The valve of the present disclosure is configured so that the amount of dead space is minimised. The filter can be located directly above the valve with only a minimal amount of spacing between these components, as the one-way valve 150 includes a resiliently deformable member fixed in position relative to the flow channel. The deformable member comprises a sealing surface which, under normal operating pressures, is urged into contact with a mating seat to inhibit flow. At elevated pressures exceeding a defined threshold, the sealing surface elastically deforms (without translation or rotation of the valve member) to create a fluid passage between the sealing surface and the seat, thereby allowing controlled leakage. The physical movement required from the valve to open to create the fluid passage is very small, and the valve can be configured within the device to add minimal dead space by virtue of the valve design being such that it can be tightly constrained with adjacent components whilst achieving effective sealing at rest, and open sufficient fluid passage on operation. In the embodiment shown, the filter 110 vertically overlaps the valve 150, locating inside the perimeter wall of the valve 150.

The valve 150 is located directly at the end of the metering chamber 124, so there are no additional structural elements that create dead space in between the end of the piston and the lower surface of the valve 150.

Furthermore, in a liquid delivery device according to the disclosure, the position of the seal at the top of the metering chamber helps to ensure that during the priming stroke, atmospheric air does not leak through the mating surfaces at the nozzle seal or other downstream structural elements of the nozzle assembly, as the partial vacuum ends at the one-way valve/seal and the downstream elements are isolated from the pressure difference.

As noted above, the valve 150 is constrained by the rigid walls of the upper tube housing 108. This action has the further advantage that a certain minimum level of pressure is required in order to initiate opening of the valve 150. That is, the valve 150 is acting as a pressure responsive check valve, and will only open to allow flow in one direction once the pressure is at or above a certain threshold level. Gradual building of pressure tends to have the effect in use that larger droplets are formed initially, until the pressure reaches full operational force. At full operational force the pressure then provides more desirable small and even-size droplets. The valve arrangement as described and shown acts to assist with achieving pressure at full operational force as quickly as possible ('burst pressure') and to overcome the ‘start-up effect’ of certain already-known types of nebulisers where pressure builds up gradually initially. With the valve held closed until a threshold pressure is reached, this helps to provide a quicker ‘ramp up phase’, and therefore a more consistent droplet size. When the pressure reduces, the valve 150 closes quickly—there is rapid ramp down of delivery pressure ('ramp clipping'). It should be noted that with hysteresis the level for closing could be different than the threshold opening level. Achieving full operational force as quickly as possible and then rapid ramp down of delivery pressure greatly assists with forming and delivering only or almost entirely only small and evenly-sized droplets. The pressure clipping values will depend on the nozzle geometry and formulation characteristics such as viscosity and surface tension.

This is especially useful for delivery of lower volumes of medication—ramp up and tail effects are less pronounced and therefore have a smaller impact.

In a nebuliser with which the present embodiments of valve are intended for use, the usual operating pressure is around 25,000 kPa. However, the device is still capable of operating at much lower pressures. Aerosol sprays of reasonable size and droplet consistency will be created with an operating pressure of substantially 4,000 kPa (the minimum viable operating pressure), and doses can still be dispensed at pressures lower than this.

The valve of this embodiment (and the other embodiments described below) can be configured so that it will open at substantially 50% of the minimum viable operating pressure—at a pressure of substantially 2,000 kPa for the embodiments described and shown.

Having a valve that is held closed until a threshold pressure is reached helps to provide a quicker ‘ramp up phase’, even if the threshold pressure is very low (e.g. 2,000 kPa as noted above) and limits the delivery of liquid through the nozzle until it has reached sufficient pressure to generate an aerosol spray.

However, in order to produce a more optimal spray (more optimal size and droplet consistency), the valve (of this embodiment and the others described below) is more usually configured so as to have an opening pressure of substantially 50% of the usual operating pressure—that is, an opening pressure of substantially 12,500 kPa and limits the delivery of liquid through the nozzle until it has reached sufficient pressure to generate a more optimal aerosol spray.

For other types of nozzle, such as for example Rayleigh plate nozzles, the opening pressure can be much lower—the burst pressure for a more open valve solution such as a Rayleigh jet nozzle is any pressure greater than 400 kPa.

The use of a one-way valve according to the disclosure helps to ensure that a correct dosage is administered each time the device is used, and that substantially all or most of the medicament in the reservoir can be used. The one-way valve also helps to shut off the open route to the air, thus preventing air ingress through that route during priming, but also during storage/at rest. It also prevents or reduces seepage of liquid out through the nozzle when not in use/storage.

A further advantage of the disclosed embodiments is that the valve creates a seal that closes off the pathway from the top or exit of the capillary tube, to the exit of the nozzle assembly (the external environment). This has the advantage that the liquid reservoir and the capillary tube are closed off from the external environment, and evaporation of the contents of the inhaler from the nozzle is significantly reduced. Further to this, the longer-term stability performance of the inhaler is enhanced, as the valve helps to prevent the contents of the device from contact with air, and oxidation.

Locating the valve outside the capillary tube also assists with overcoming capillary effects that can still be present even if a one-way valve is present but is located in the capillary tube (and which can lead to deposits of non-volatile residues at the nozzle exit), and allows more effective valves to be used.

FIG. 13 shows a cross-section view of a device that contains the valve 150 and upper tube housing 108 in use. The flow path from the metering chamber to the nozzle is shown by arrows 160.

As can be seen on this figure, the outer part of the lower face of the seal 150 seals against the lower face of the recess 108a, and against the internal surface of the side walls of the recess 108a, acting as a gasket and maintaining a sealing engagement between the seal 150 and the upper tube housing 108. As can be seen in e.g. FIGS. 9a and 9b, the lower surface of the filter holder 109 is also in contact with the top of the seal 150 at the edge of the central recess of the filter holder 109 (the recess that holds the filter 110), the seal acting as a gasket and maintaining a sealing engagement between the seal 150 and the filter holder 109. That is, the filter holder 109 is in contact with the upper surfaces of the Y-shaped connecting members 150c, leaving the upper surface of the central sealing portion 150b free/not in contact with the filter holder (the upper surface of the central sealing portion 150b is slightly below the top surfaces of the Y-shaped connecting members 150c. The middle of the seal—the central sealing portion 150b—is not held or restrained from upwards deformation, and this space or distance also provides a small amount of clearance to allow for tolerance stack and fluid flow access to the whole of the filter surface area for optimised filtration with minimal unnecessary dead space. The area directly below the central sealing portion 150b is the metering chamber.

The seal 150 is directly adjacent to the metering chamber, and forms a pressure-retaining seal within the upper tube housing 108, at the top of the metering chamber. However, the central sealing portion 150b has no functional mating sealing surface with the upper tube housing 108, as the central sealing portion 150b is substantially the same size as, and axially aligned with, the passage through the upper tube housing 108. As can be seen from the figures, the total diameter of the recess 108a is greater than that of the central passage which passes through the upper tube housing 108. The metering chamber/passage is sealed by way of the lower surface and outer wall of the seal main body 150a and the inner surface of the recess 108a,with the central sealing portion 150b locating over the passage to block the passage. The apertures 150d are located diametrically outside of the central sealing portion 150b as shown in FIGS. 10a and 10b. Therefore, in order for the apertures 150d to be accessed, and for flow to pass through the apertures, the central portion 150b has to deform in order for flow to pass out of the passage.

When the internal pressure within the metering chamber exceeds a defined threshold, the central sealing portion 150b elastically deforms, opening a flow path through the apertures 150d and enabling fluid in the metering chamber to access these controlled flow apertures. The fluid is thereby allowed to flow through these apertures past the seal 150, whilst the gasket function between the side walls of the recess 108a and the filter holder 109 remains uncompromised.

Upon reduction or removal of the internal chamber pressure, the sealing element substantially reverts towards its original, undeformed position, thereby re-sealing the chamber and occluding the apertures 150d to prevent further fluid communication.

For the preferred embodiment, the amount of fluid that passes through the valve 150 when each dose is dispensed is substantially 10 Cubic Micrometres (10 μm³), but can be up to a maximum of 200 Cubic Micrometres (200 μm³).

Due to the elastic properties and inherent stiffness of the material that forms the seal 150 (which is a unitary member), a degree of residual pressure persists within the metering chamber following resealing.

The seal 150 is held tightly around the outside of the seal (by the wall of the upper tube housing 108 that forms the recess 108a), so as to act as a gasket which is permanently in a sealing arrangement. The contact area between the seal 150 and the upper tube housing 108 remains unchanged/constant during use—that is, there is no movement of the seal 150 as a whole, relative to the upper tube housing 108—the seal 150 remains in position in the recess 108a. The contact between the connecting members 150c and the filter holder 109 ensures that the seal 150 is sandwiched between the upper tube housing 108 and the filter holder 109, so that the seal cannot move and is held in position, but can deform to allow fluid to pass through.

This contact helps to provide a sealing force on the mating sealing surfaces around the circumference of the passage opening to/from the metering chamber, and to assist with preventing/limiting movement of the seal under pressure, and opening of the fluid channel on device actuation. The central sealing portion 150b is not restrained in position. This allows for tolerance stack and fluid flow access to the whole of the filter surface area for optimised filtration while minimising unnecessary dead space.

The degree of deformation of the central sealing portion 150b, and hence the extent to which the apertures 150d (which form controlled flow channels) are opened, is influenced by the flow resistance imposed by the elements of the overall system downstream of the valve 150.

In configurations incorporating a highly restrictive nozzle such as one with a total cross-sectional area of approximately 80 μm² and requiring operational pressures in the order of 100-300 bar, the resulting backpressure substantially limits the volumetric flow rate and, in turn, constrains the deformation of the one-way valve 150.

The relationship between nozzle cross-sectional area and the extent of valve opening are strongly linked. Although linked, they may not be strictly linear. While reduced downstream area leads to higher backpressure and a correspondingly smaller degree of deflection of the central sealing portion 150b under steady-state conditions, additional transient deformation of the valve 150 may occur during startup or initial pressurisation. This is due to the need to overcome static resistance effects such as initial seal compression, material inertia, and the resistance of the fluid to first movement, including potential surface tension and compressibility effects.

As such, the system exhibits a dynamic interaction between upstream pressure, compliance of the one-way valve, and downstream restriction. The sealing element—the central sealing portion 150b—may exhibit a greater degree of opening during pressure ramp-up, with subsequent partial relaxation once flow is established. This behaviour contributes to a passive flow modulation mechanism, wherein the extent of deflection and the activation of the flow channels are governed not only by the applied chamber pressure, but also by the hydraulic impedance of the downstream nozzle or outlet.

The dynamic interaction between the chamber pressure, elasticity of the one-way valve, and downstream hydraulic resistance can result in a transient flow area through the controlled channels that is between approximately 50% and 200% of the downstream nozzle cross-sectional area, depending on the system dynamics and the pressure ramp profile.

A one-way valve according to the disclosure, used with an inhaler according to the embodiment described, only opens by an area similar to the nozzle jet area/nozzle cross sectional area.

1. A one-way valve for a liquid delivery device, comprising:
a main body configured to locate and seal within a passage within the liquid delivery device;
a sealing portion configured so that in a first state fluid flow through the passage is substantially blocked, and in a second state fluid can flow through the passage;
characterised in that
the main body and sealing portion comprise parts of a unitary item, the sealing portion configured to deform from the first state to the second state in use, the main body configured so as to remain substantially in position and substantially undeformed.

2. A one-way valve for a liquid delivery device as claimed in claim 1 wherein the valve further comprises a plurality of connecting members configured to extend between the main body and the sealing portion to bias the blocking member towards the first position, a plurality of apertures formed between the connecting members, the apertures blocked in the first state and open in the second state.

3. A one-way valve for a liquid delivery device as claimed in claim 1 wherein the main body comprises a ring-shaped body and the central sealing portion comprises a disc-shaped member, the main body substantially surrounding and enclosing the sealing portion, the central sealing portion sized to substantially fully fill the lower end of the ring-shaped main body.

4. A one-way valve for a liquid delivery device as claimed in claim 3 wherein the central sealing portion has a height or thickness substantially one-third the height of the main body.

5. A one-way valve for a liquid delivery device as claimed in claim 4 wherein the lower face/side of the central sealing portion is substantially aligned with and in the same plane as the lower end of the main body.

6. A one-way valve for a liquid delivery device as claimed in claim 3 wherein the upper and lower faces of the disc-shaped member are substantially flat and parallel to one another.

7. A one-way valve for a liquid delivery device as claimed in claim 2 wherein the connecting members comprise four Y-shaped pillars that extend in between and connect between the central sealing portion and the ring-shaped main body, the pillars located at equally-spaced intervals around the inside of the main body.

8. A liquid delivery device, comprising:
a lower housing part configured to contain a reservoir;
an upper housing part configured to contain a nozzle assembly, the nozzle assembly comprising an upper tube housing,
a passage extending between the reservoir and the nozzle assembly to in use deliver liquid from the reservoir to the nozzle assembly, at least part of the passage formed through the upper tube housing, the upper part of the passage within the upper tube housing forming a metering chamber;
the nozzle assembly configured to convert liquid received from the metering chamber to a droplet spray at the outer or downstream end of the nozzle assembly for delivery to a user;
characterised in that
the liquid delivery device further comprises a one-way valve according to claim 1;
the upper tube housing comprises a recess at the upper end of the upper tube housing, the recess configured to receive and substantially fully confine the outer side or sides of the one-way valve.

9. A liquid delivery device as claimed in claim 8 wherein the valve is located substantially directly at the upper end of the metering chamber.

10. A liquid delivery device as claimed in claim 8 wherein the recess of the upper tube housing and nozzle assembly are configured so that the end of the nozzle assembly locates into the recess.

11. A liquid delivery device as claimed in claim 8 wherein the valve and nozzle assembly are further mutually configured so that the valve is confined within the recess by contact with the nozzle assembly.

12. A liquid delivery device as claimed in claim 8 wherein the liquid delivery device further comprises a spring configured to provide pressure to liquid within the metering chamber, the valve configured to deform to allow substantially 80 Cubic Micrometres to pass therethrough.

13. A liquid delivery device as claimed in claim 8 wherein the one-way valve and nozzle assembly are mutually configured so that the one-way valve only opens by an area substantially the same as the nozzle jet area/nozzle cross sectional area.
